(19) United States
(12) Patent Application Publication
(10) Pub. No.: US 2008/0058845 A1
(43) Pub. Date: Mar. 6, 2008

(54) Ultrasonic Surgical Operation Instrument

(76) Inventors: Koh Shimizu, Tokyo (JP); Shinya Masuda, Yokohama (JP); Kenichi Kimura, Tokyo (JP)

Correspondence Address: OSTROLENK FABER GERB & SOFFEN, 1180 AVENUE OF THE AMERICAS, NEW YORK, NY, 100368403, US

(21) Appl. No.: 11/570,881

(22) PCT Filed: Jun. 10, 2005
(86) PCT No.: PCT/JP05/10723

§ 371 Date: Dec. 18, 2006

(30) Foreign Application Priority Data
Jun. 10, 2005 (JP) 2004-178914

(51) Int. Cl. A61B 17/00

(52) U.S. Cl. 606/169; 604/22; 606/205

(57) ABSTRACT

An ultrasonic surgical operation instrument characterized by comprising a treating section for treating living tissues using ultrasonic vibrations, a heat sensor disposed in the treating section for detecting the temperature of the treating section, and a control unit for controlling the temperature of the treating section on the basis of temperature information detected by the heat sensor.

### CROSS REFERENCE TO RELATED APPLICATIONS

This is a Continuation Application of PCT Application No. PCT/JP2005/010723, filed Jun. 10, 2005, which was not published under PCT Article 21(2) in English.

This application is based upon and claims the benefit of priority from a prior Japanese Patent Application, Japanese Patent Application No. 2004-178914, filed Jun. 16, 2004, the entire contents of which are incorporated herein by reference.

### BACKGROUND OF THE INVENTION

1. Field of the Invention

The present invention relates to an ultrasonic surgical operation instrument that grasps living tissues and performs ultrasonic treatments such as incisions, coagulations and the like on living tissues.

2. Description of the Related Art

Ultrasonic surgical operation instruments are instruments that perform ultrasonic treatments such as incisions, coagulations and the like on living tissues.

In conventional ultrasonic surgical operation instruments, an ultrasonic vibrator is disposed in an operating unit, which is operated by an operator, and an ultrasonic probe is disposed on the tip. The ultrasonic probe transmits ultrasonic vibrations generated by the ultrasonic vibrator and performs the treatment on living tissues.

In addition, a rotatable jaw is mounted opposite to the ultrasonic probe. A movable handle for controlling the opening and closing operations of the jaw is provided in the operating unit.

Accordingly, in the above conventional ultrasonic surgical operation instrument, living tissues are grasped by the ultrasonic probe and the jaw; the jaw opens and closes with respect to the ultrasonic probe via the operation of the movable handle.

In the conventional ultrasonic surgical operation instrument, the ultrasonic vibrations from the ultrasonic vibrator are transmitted to the ultrasonic probe while grasping living tissues. Thereby, treatments such as incisions, coagulations and the like are given to the grasped living tissues.

Japanese Patent Application Publication No. 9-299381 and Japanese Patent Application Publication No. 11-70118 disclose the conventional ultrasonic surgical operation instruments as above.

In the ultrasonic surgical operation instrument disclosed in Japanese Patent Application Publication No. 9-299381, the ultrasonic output from the ultrasonic vibrator is set to be higher than that for normal operation when starting the ultrasonic treatment in order to enhance the response of the ultrasonic treatment. Also, this ultrasonic surgical operation instrument is configured to switch and control the operation status after starting the ultrasonic treatment so that the ultrasonic output from the ultrasonic vibrator will be the setting output value.

In the ultrasonic surgical operation instrument disclosed in Japanese Patent Application Publication No. 11-70118, a constant current control is performed on the current supplied to the ultrasonic vibrator in order to reduce the load on the ultrasonic vibrator and the voltage applied to the ultrasonic vibrator is monitored. On the basis of this monitoring, the ultrasonic surgical operation instrument is configured to switch the driving method (constant power drive and constant voltage drive) from constant current control to an energy limiting control when the monitored voltage reaches the limit voltage.

### SUMMARY OF THE INVENTION

The ultrasonic surgical operation instrument according to the present invention comprises a treatment unit for performing treatments on living tissue with ultrasonic vibrations, a heat sensor disposed in the treatment unit for detecting the temperature of this treatment unit, and control unit for controlling the temperature of the treatment unit on the basis of the temperature information detected by the heat sensor.

### BRIEF DESCRIPTION OF THE DRAWINGS

FIG. 1 is an overall view showing an ultrasonic surgical operation instrument according to the first embodiment;

FIG. 2 is a schematic cross-sectional view showing a configuration of the ultrasonic treatment tool shown in FIG. 1;

FIG. 3 is a block diagram for a circuit of the ultrasonic surgical operation instrument in FIG. 1;

FIG. 4 is a flowchart showing an operation of an incision mode according to the first embodiment;

FIG. 5 is a flowchart showing an operation of a coagulation mode according to the first embodiment;

FIG. 6 is an overall view showing an ultrasonic surgical operation instrument according to the second embodiment;

FIG. 7 is a schematic cross-sectional view showing a configuration of the ultrasonic treatment tool in FIG. 6;

FIG. 8 is a block diagram for a circuit of the ultrasonic surgical operation instrument in FIG. 6;

FIG. 9 is a flowchart showing the operation of an incision mode according to the second embodiment;

FIG. 10 is a flowchart showing the operation of a coagulation mode according to the second embodiment;

FIG. 11 is an overall view showing an ultrasonic surgical operation instrument according to the third embodiment;

FIG. 12 is a schematic cross-sectional view showing a configuration of the ultrasonic treatment tool in FIG. 11;

FIG. 13 is a block diagram for a circuit of the ultrasonic surgical operation instrument in FIG. 11;

FIG. 14 is a flowchart showing the operation of an incision mode according to the third embodiment; and

FIG. 15 is a flowchart showing the operation of a coagulation mode according to the third embodiment.

### DESCRIPTION OF THE PREFERRED EMBODIMENTS

The present invention provides an ultrasonic surgical operation instrument in which an operator can unconsciously control energy applied to living tissue and can prevent the temperature applied to the living tissue from becoming too high.

Hereinbelow, embodiments of the present invention will be explained by referring to the drawings.

### First Embodiment

The ultrasonic surgical operation instrument disclosed in Japanese Patent Application Publication No. 9-299381 above switches the ultrasonic output value after a prescribed time period elapses. Accordingly, in the ultrasonic surgical operation instrument in the above document, there is a probability that constant effects will not be attained if the temperature at the grasped part at the time of an incision or the temperature for the coagulation of living tissue is not constant.

Also, in the ultrasonic surgical operation instrument disclosed in Japanese Patent Application Publication No. 11-70118 above, while performing the treatment the operator or the assistant always pays attention to the load state on the ultrasonic vibrator, which is reported from the main body of the instrument. Also, the ultrasonic surgical operation instrument in the above document requires the application of electric power to the living tissue to be controlled by the operator himself/herself on the basis of the load status of the ultrasonic vibrator.

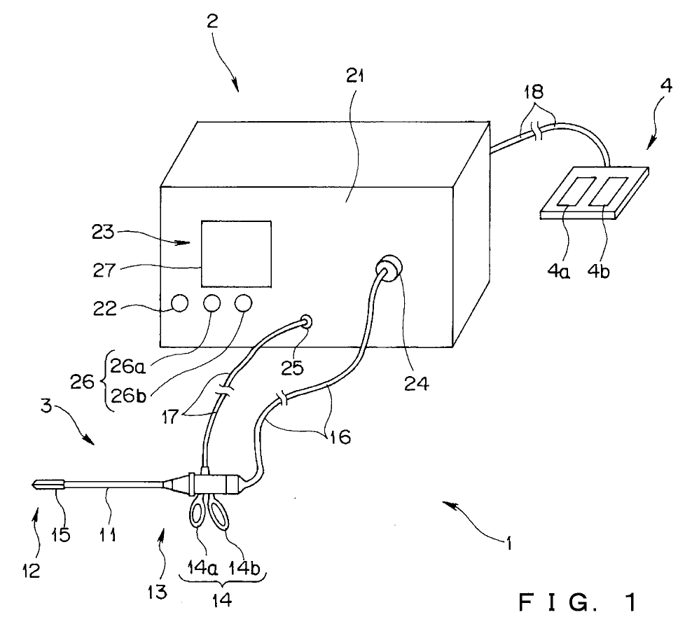

FIG. 1 is an overall view showing an ultrasonic surgical operation instrument according to the first embodiment. As shown in FIG. 1, the ultrasonic surgical operation instrument 1 according to the present embodiment has a configuration in which an ultrasonic treatment tool 3 and a foot switch 4 are connected to an ultrasonic surgical operation instrument body (hereinafter, simply referred to as instrument body) 2.

The foot switch 4 comprises an incision switch 4a and a coagulation switch 4b. The incision switch 4a is for executing an incision mode. The coagulation switch 4b is for executing a coagulation mode.

In the ultrasonic treatment tool 3, a treatment unit 12 is disposed on the tip portion of an elongated sheath shaped insertion-unit jacket 11 and a handpiece 13 to be operated by an operator is disposed at the proximal end. The handpiece 13 includes an ultrasonic vibrator for generating ultrasonic vibrations; this ultrasonic vibrator will be described later. Also, an operation handle 14 is provided on the handpiece 13 for operating the treatment unit 12.

The operation handle 14 comprises a fixed handle 14a and a movable handle 14b. The fixed handle 14a is integrated with the handpiece 13. The movable handle 14b is provided in an openable and closable state with respect to the handpiece 13.

An ultrasonic probe 15 for transmitting the ultrasonic vibrations from the ultrasonic vibrator to the treatment unit 12 is disposed in the insertion-unit jacket 11. A tip portion of this ultrasonic probe 15 is exposed from a tip portion of the insertion-unit jacket 11.

An ultrasonic cable 16 extends to the handpiece 13 and is electrically connected to the ultrasonic vibrator. The ultrasonic cable 16 is detachably connected to the instrument body 2. Also, a heat sensor cable 17 electrically connected to a heat sensor is extended to the handpiece 13; the heat sensor will be described later. This heat sensor cable 17 is detachably connected to the instrument body 2.

A power switch 22, an operation display unit 23, an ultrasonic cable connection unit 24, and a heat sensor cable connection unit 25 are arranged on the front panel 21 of the instrument body 2. A foot switch cable 18 for the foot switch 4 is detachably connected to the rear panel of the instrument body 2.

An ultrasonic cable 16 of the ultrasonic treatment tool 3 is detachably connected to the ultrasonic cable connection unit 24. Also, the heat sensor cable 17 of the ultrasonic treatment tool 3 is detachably connected to the heat sensor cable connection unit 25.

Also, setting switches 26 and a display section 27 are provided to the operation display unit 23. The setting switches 26 are for setting the magnitude of the ultrasonic output for normal operation when performing an ultrasonic treatment. The display section 27 digitally displays the magnitude of the ultrasonic output set by these setting switches 26.

The setting switches 26 include an output increase switch 26a and an output decrease switch 26b. The output increase switch 26a is for changing (increasing) the magnitude of the ultrasonic output. The output decrease switch 26b is for changing (decreasing) the magnitude of the ultrasonic output.

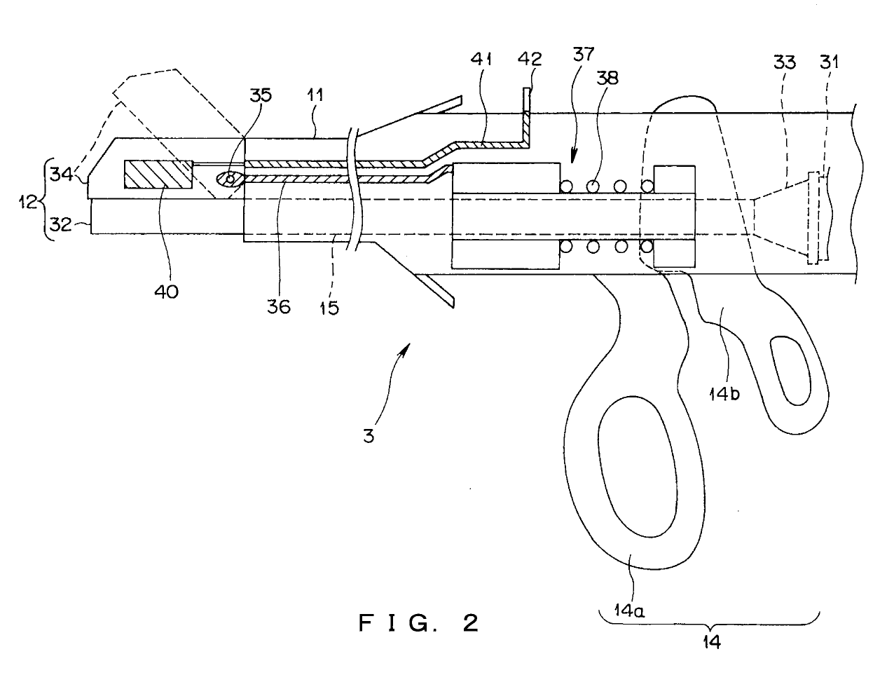

Next, the configuration of the instrument body 2 will be explained in detail by referring to FIG. 2.

FIG. 2 is a schematic cross-sectional view showing the configuration of the ultrasonic treatment tool in FIG. 1. As shown in FIG. 2, an ultrasonic vibrator 31 is provided to the proximal end of the ultrasonic probe 15 in the ultrasonic treatment tool 3. Also, a tip treatment unit 32 is provided to the ultrasonic probe 15. The tip treatment unit 32 has an elliptical cross section.

A horn 33 for amplifying amplitude is connected to the tip side of the ultrasonic vibrator 31. The tip side of the horn 33 is fixed to the proximal side of the ultrasonic probe 15.

The ultrasonic vibrator 31 generates ultrasonic vibrations when a drive signal is sent from the instrument body 2 via the ultrasonic cable 16. The ultrasonic vibration generated by the ultrasonic vibrator 31 is transmitted to the ultrasonic probe 15 after being amplified by the horn 33. The ultrasonic vibration transmitted to the ultrasonic probe 15 is transmitted to the tip treatment unit 32 and applied to the living tissue.

A jaw 34 that is openable/closable is provided in the treatment unit 12 of the ultrasonic treatment tool 3. This jaw 34 is supported by the tip portion of the insertion-unit-jacket 11 in a state in which the jaw 34 is rotatable about a support shaft 35. The tip portion of an operation rod 36 is connected to the support shaft 35. This operation rod 36 is disposed in the insertion-unit-jacket 11. A back portion of the operation rod 36 is connected to an operation force adjustment unit 37.

This operation force adjustment unit 37 is connected to the movable handle 14b. The operation force adjustment unit 37 adjusts the operation force from the movable handle 14b by the biasing force of a coil spring 38 and moves the operation rod 36 back and forth. The operation rod 36 that is moved back and forth opens and closes the jaw 34 via the support shaft 35.

In the present embodiment, when the operation handle 14 is gripped with fingers other than the thumb on the fixed handle 14a and the thumb on the movable handle 14b, the operation force adjustment unit 37 pushes the operation rod 36 toward the tip portion while adjusting the operation force from the movable handle 14b, and the jaw 34 closes with respect to the tip treatment unit 32 of the ultrasonic probe 15 rotating about the support shaft 35.

A heat sensor 40 is provided on the tip portion of the jaw 34, which can be the hottest portion. This heat sensor 40 may be, for example, a thermocouple or a thermostat.

A heat sensor cable 41 is extended to the heat sensor 40. This heat sensor cable 41 is connected to a heat sensor terminal 42. A heat sensor cable 17 is detachably connected to this heat sensor terminal 42. The heat sensor 40 detects the temperature of the jaw 34 and transmits the detected temperature information to the instrument body 2. The frequency of sampling by this heat sensor 40 may be, for example, ten times a second or higher.

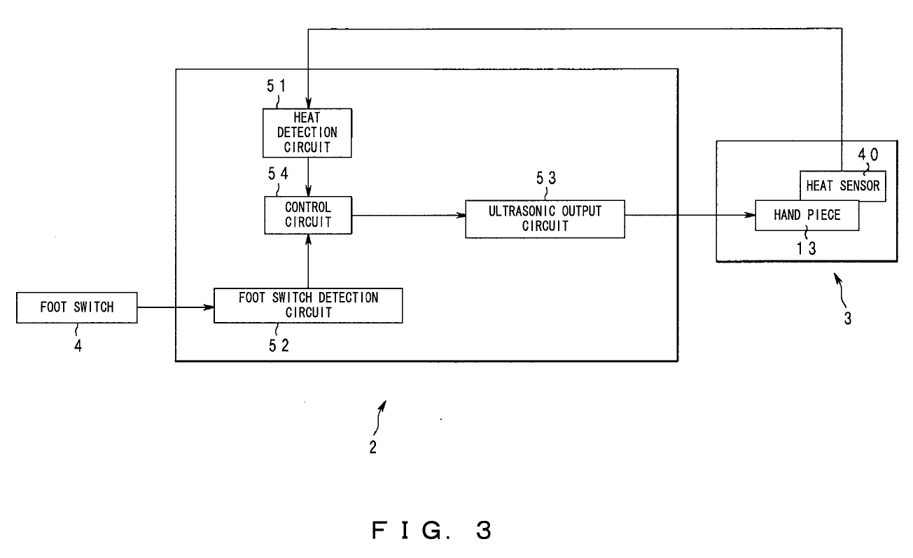

Next, the configuration of the instrument body 2 will be explained in detail by referring to FIG. 3.

FIG. 3 is a block diagram for a circuit of the ultrasonic surgical operation instrument in FIG. 1. As shown in FIG. 3, the instrument body 2 comprises a heat detection circuit 51, a foot switch detection circuit 52, an ultrasonic output circuit 53, and a control circuit 54.

The heat detection circuit 51 detects the temperature of the jaw 34 by obtaining temperature information from the heat sensor 40 of the ultrasonic treatment tool 3. The heat detection circuit 51 outputs the detected temperature information of the jaw 34 to the control circuit 54.

The foot switch detection circuit 52 receives an on/off signal from the foot switch 4 and outputs the received on/off signal to the control circuit 54.

The ultrasonic output circuit 53 receives the on/off signal and the ultrasonic amplitude value signal from the control circuit 54, and outputs a drive signal for controlling the drive of the ultrasonic vibrator 31 of the ultrasonic treatment tool 3.

The control circuit 54 controls the ultrasonic output circuit 53 on the basis of the on/off signal output from the foot switch detection circuit 52 and on the basis of the temperature information of the jaw 34 output from the heat detection circuit 51.

In other words, the control circuit 54 receives the input of the on/off signal of the foot switch 4 and the temperature information and outputs the on/off signal and the ultrasonic amplitude value signal to the ultrasonic output circuit 53. In the present embodiment, a configuration is employed in which the treatment unit 12 (jaw 34) maintains a prescribed temperature at which the living tissue is not incised but is coagulated in the coagulation mode. The operation of the control circuit 54 will be explained by referring to the flowchart, which will be explained later.

An ultrasonic surgical operation instrument 1 configured as above can efficiently perform ultrasonic treatments such as incisions, coagulations and the like to living tissues.

The operator turns on the power switch 22 of the instrument body 2. Then, the heat sensor 40 of the ultrasonic treatment tool 3 detects the temperature of the jaw 34 and starts transmitting the detected temperature information to the instrument body 2 via the heat sensor cable 17.

The operator places his/her fingers other than his/her thumb on the fixed handle 14a, places his/her thumb on the movable handle 14b, and grips the operation handle 14 of the ultrasonic treatment tool 3 while resisting the biasing force of a coil spring 38. In the ultrasonic treatment tool 3, the operation force adjustment unit 37 pushes the operation rod 36 toward the tip side while adjusting the operation force from the movable handle 14b via operations on the handle by the operator.

The force transmitted by the forward motion of the operation rod 36 acts on the jaw 34 to close the jaw 34 rotating about the support shaft 35. Then, the jaw 34 closes with respect to the tip treatment unit 32 of the ultrasonic probe 15 while pinching living tissue between the jaw 34 itself and the tip treatment unit 32 of the ultrasonic probe 15. Thereby, the jaw 34 grasps the living tissue between the jaw 34 itself and the tip treatment unit 32.

In this state, the operator depresses the foot switch 4 and performs the ultrasonic treatment on the living tissue grasped between the jaw 34 and the tip treatment unit 32 of the ultrasonic probe 15. The grasped living tissue receives ultrasonic operations such as incisions, coagulations, or the like via friction heat between the living tissue itself and the tip treatment unit 32, which vibrates at high speed.

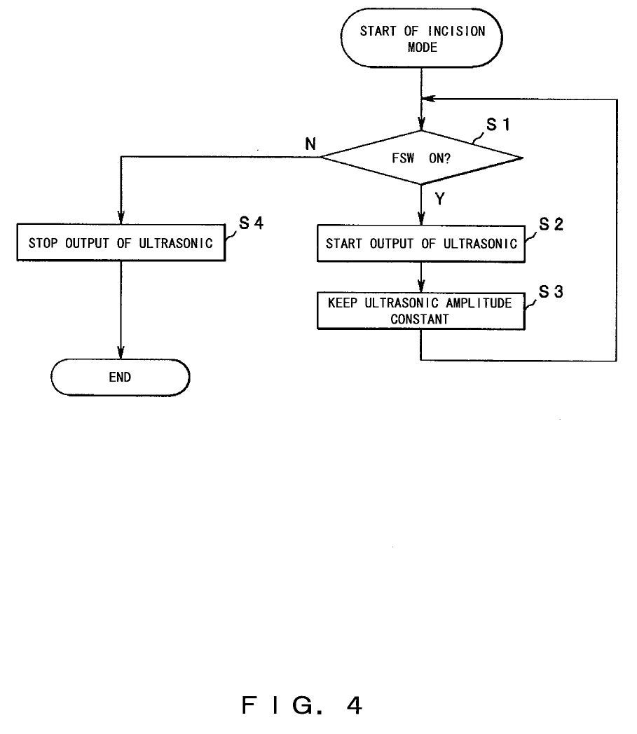

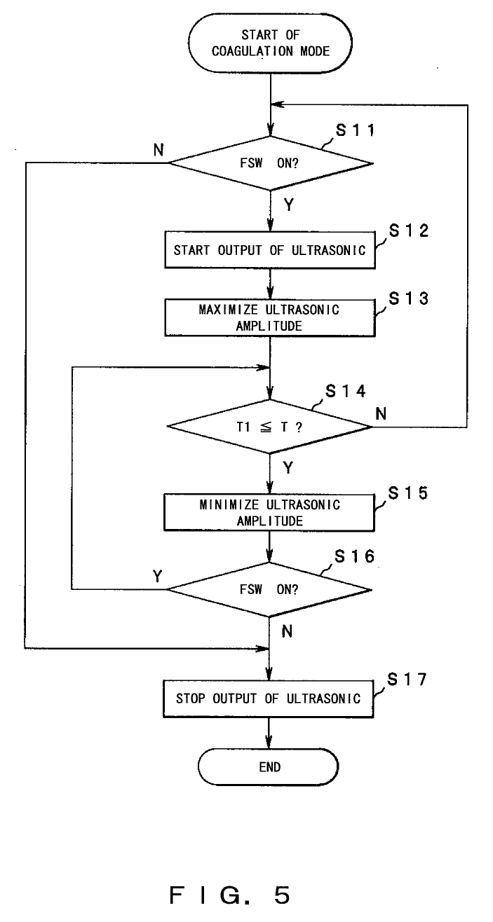

Then, the ultrasonic surgical operation instrument 1 operates according to the flowchart of FIG. 4 or FIG. 5. First, the incision mode is explained.

FIG. 4 is a flowchart showing the operation of the incision mode according to the first embodiment. When the operator depresses the incision switch 4a of the foot switch 4, the foot switch 4 outputs an incision-on signal via the foot switch cable 18.

In the ultrasonic surgical operation instrument 1, the control circuit 54 starts control as shown in FIG. 4.

The control circuit 54 determines whether or not the incision switch 4a of the foot switch 4 is pressed (step S1). The control circuit 54 terminates the process of this flowchart when the incision-on signal is not received from the foot switch 4 via the foot switch detection circuit 52.

When the incision-on signal is received from the foot switch 4 via the foot switch detection circuit 52, the control circuit 54 outputs an on signal to the ultrasonic output circuit 53 and starts outputting the ultrasonic (step S2).

Then, the control circuit 54 receives the temperature information of the jaw 34 via the heat detection circuit 51. On the basis of the temperature information received from the jaw 34, the control circuit 54 outputs the ultrasonic amplitude value signal such that the ultrasonic amplitude of the drive signal output from the ultrasonic output circuit 53 is constant.

The ultrasonic output circuit 53 outputs the drive signal that makes the ultrasonic amplitude constant; this drive signal is based on the ultrasonic amplitude value signal from the control circuit 54.

The drive signal from the instrument body 2 is transmitted to the ultrasonic vibrator 31 of the ultrasonic treatment tool 3 via the ultrasonic cable 16 and drives the ultrasonic vibrator 31.

Receiving the drive signal, the ultrasonic vibrator 31 generates ultrasonic vibrations such that the ultrasonic amplitude is constant (step S3). This ultrasonic vibration is transmitted to the tip treatment unit 32 of the ultrasonic probe 15. The tip treatment unit 32 vibrates at high speed such that the ultrasonic amplitude is constant. The grasped living tissue is incised due to the friction heat between the living tissue itself and the tip treatment unit 32 vibrating at high speed.

During this incision, the operator keeps his/her foot on the incision switch 4a of the foot switch 4. When the operator lifts his/her foot away from the incision switch 4a of the foot switch 4, the foot switch 4 outputs an incision-off signal via the foot switch cable 18.

When the incision-off signal is received from the foot switch 4 via the foot switch detection circuit 52, the control circuit 54 outputs the off signal to the ultrasonic output circuit 53.

The ultrasonic output circuit 53 stops the output of the drive signal to the ultrasonic vibrator 31 when it receives the off signal from the control circuit 54 (step S4). The ultrasonic vibrator 31 stops the ultrasonic vibration and the ultrasonic treatment terminates.

In the above configuration, the ultrasonic amplitude is always constant in the ultrasonic surgical operation instrument 1 in the incision mode.

Next, the coagulation mode is explained.

FIG. 5 is a flowchart showing the operation of the coagulation mode according to the first embodiment. When the operator depresses the coagulation switch 4b of the foot switch 4, the foot switch 4 outputs a coagulation-on signal via the foot switch cable 18.

In the ultrasonic surgical operation instrument 1, the control circuit 54 starts control as shown in FIG. 5. The control circuit 54 determines whether or not the coagulation switch 4b of the foot switch 4 is pressed (step S11). The control circuit 54 terminates the process depicted by this flowchart when the coagulation-on signal is not received from the foot switch 4 via the foot switch detection circuit 52.

When the coagulation-on signal is received from the foot switch 4 via the foot switch detection circuit 52, the control circuit 54 outputs an on signal to the ultrasonic output circuit 53 and starts outputting the ultrasonic (step S12).

Then, the control circuit 54 generates a ultrasonic amplitude value signal such that the ultrasonic amplitude becomes greatest immediately after the start of output of the ultrasonic; the control circuit 54 then outputs the signal to the ultrasonic output circuit 53.

On the basis of the ultrasonic amplitude value signal from the control circuit 54, the ultrasonic output circuit 53 outputs the drive signal such that the ultrasonic amplitude reaches its peak.

The drive signal from the instrument body 2 is transmitted to the ultrasonic vibrator 31 of the ultrasonic treatment tool 3 via the ultrasonic cable 16 and drives the ultrasonic vibrator 31.

Receiving the drive signal, the ultrasonic vibrator 31 generates ultrasonic vibrations such that the ultrasonic amplitude reaches its peak (step S13). This ultrasonic vibration is transmitted to the tip treatment unit 32 of the ultrasonic probe 15. The tip treatment unit 32 vibrates at high speed such that the ultrasonic amplitude reaches its peak. The grasped living tissue begins to coagulate due to the friction heat between the living tissue itself and the tip treatment unit 32 vibrating at high speed.

Then, the control circuit 54 monitors the temperature T of the treatment unit 12 during the coagulation and controls the ultrasonic amplitude such that the temperature of the treatment unit 12 is around a prescribed temperature T1 (around 120° C.). The temperature T1 (around 120° C.) is the temperature at which the living tissue is not incised but is subject to heat denaturation.

In other words, the control circuit 54 determines whether or not the temperature T of the treatment unit 12 will become equal to or greater than the prescribed temperature T1 (around 120° C.) during the coagulation (step S14).

When the temperature T of the treatment unit 12 is lower than the temperature T1 (“N” at step S14), the control circuit 54 returns to step S11 and repeats the above steps from step S11 to step S14 until the temperature T of the treatment unit 12 becomes equal to or higher than the prescribed temperature T1 (around 120° C.).

When the temperature T of the treatment unit 12 is equal to or higher than the temperature T1 (“Y” at step S14), the control circuit 54 generates an ultrasonic amplitude value signal such that the ultrasonic amplitude attains its minimum value and outputs the generated ultrasonic amplitude value signal to the ultrasonic output circuit 53.

On the basis of the ultrasonic amplitude value signal from the control circuit 54, the ultrasonic output circuit 53 outputs the drive signal such that the ultrasonic amplitude attains its minimum value.

Receiving the drive signal, the ultrasonic vibrator 31 generates ultrasonic vibrations such that the ultrasonic amplitude attains its minimum value (step S15). The ultrasonic vibration is transmitted to the tip treatment unit 32 of the ultrasonic probe 15. The tip treatment unit 32 vibrates at a low speed such that the ultrasonic amplitude attains its minimum value.

Accordingly, in the ultrasonic treatment tool 3, the temperature T of the treatment unit 12 becomes the temperature T1 (around 120° C.).

During this coagulation, the operator keeps his/her foot on the coagulation switch 4b of the foot switch 4. When the operator lifts his/her foot away from the coagulation switch 4b of the foot switch 4, the foot switch 4 outputs a coagulation-off signal via the foot switch cable 18.

The control circuit 54 determines whether or not the coagulation switch 4b of the foot switch 4 is in the on state (step S16). The control circuit 54 repeats the above steps from step S14 to step S16 until the coagulation-off signal is received from the foot switch 4 via the foot switch detection circuit 52.

When the coagulation-off signal is received from the foot switch 4 via the foot switch detection circuit 52, the control circuit 54 outputs the off signal to the ultrasonic output circuit 53.

The ultrasonic output circuit 53 stops the output of the drive signal to the ultrasonic vibrator 31 when it receives the off signal from the control circuit 54 (step S17). The ultrasonic vibrator 31 stops the ultrasonic vibration and the ultrasonic treatment terminates.

In the above configuration, the ultrasonic surgical operation instrument 1 generates ultrasonic vibrations at high speed from the start of the ultrasonic output until the temperature reaches the prescribed temperature T1 and generates ultrasonic vibrations at low speed such that the living tissue is not incised but is coagulated due to heat denaturation when the temperature reaches temperature T1 in the coagulation mode.

Accordingly, the ultrasonic surgical operation instrument 1 can control the increase in the temperature of the treatment unit 12 and of the grasped living tissue by controlling the friction heat.

As a result of this, the ultrasonic surgical operation instrument 1 can control the energy application to the living tissue and can prevent the temperature with respect to the living tissue from becoming too high without requiring the consciousness of the operator.

### Second Embodiment

In the above first embodiment, the temperature of the treatment unit 12 is maintained at the prescribed temperature by controlling the ultrasonic amplitude value. In contrast, in a second embodiment, a configuration is employed in which the temperature of the treatment unit 12 is kept at the prescribed temperature via cooling by means of fed water/air. The aspects of the configuration other than this one are generally the same as those of the first embodiment; accordingly, the explanations thereof are omitted and like members are denoted by like symbols.

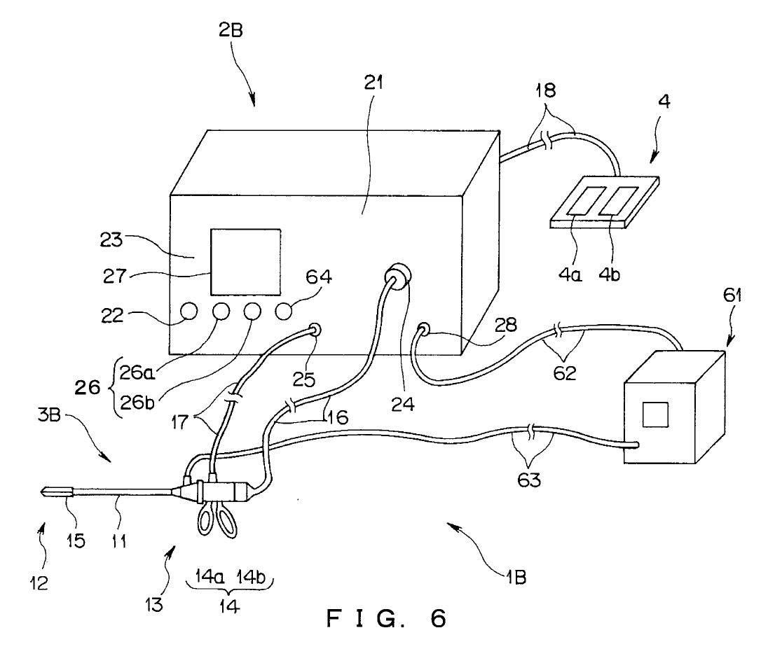

FIG. 6 is an overall view showing the ultrasonic surgical operation instrument according to the second embodiment. As shown in FIG. 6, the ultrasonic surgical operation instrument 1B according to the present embodiment comprises an air/water feeder 61. The air/water feeder 61 feeds air/water to the ultrasonic treatment tool 3B in order to cool the treatment unit 12 of the ultrasonic treatment tool 3B.

In an instrument body 2B, the air/water feeder 61 is connected to an air/water feed control cable connection unit 28 via an air/water feed control cable 62. The ultrasonic treatment tool 3B is connected to the air/water feeder 61 via an air/water feed cable 63 extending from the tip side of the handpiece.

An air/water interface switch 64 is provided to the front panel 21 of the instrument body 2B. By pressing this air/water interface switch 64, the instrument body 2B can turn on/off the interface of the air/water feed.

As will be described later, the air/water feeder 61 is controlled by the instrument body 2B via the air/water feed control cable 62 and feeds air/water to the ultrasonic probe 15 via the air/water feed cable 63. The air/water feeder 61 comprises a water feed bottle (not shown) for feeding water.

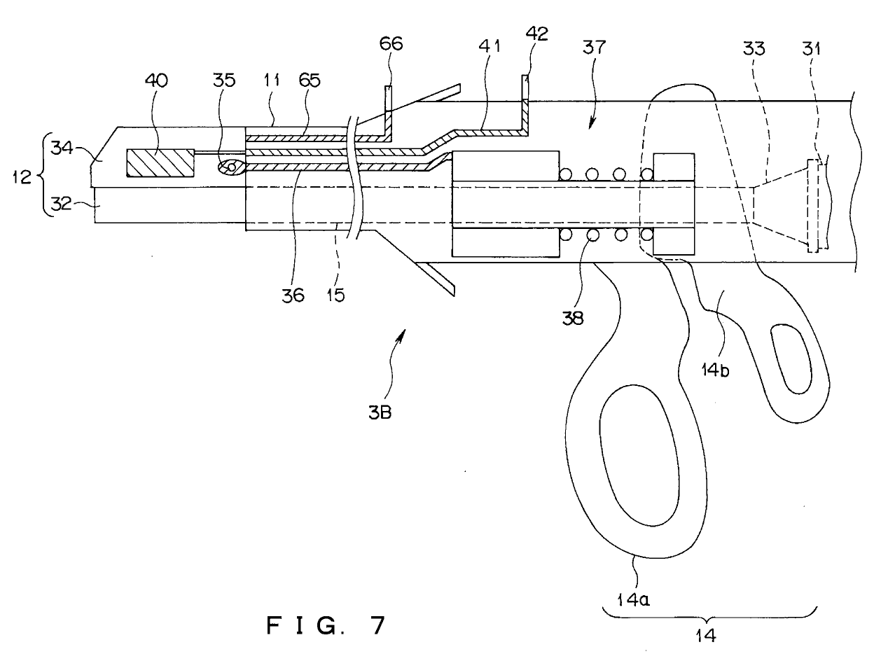

FIG. 7 is a schematic cross sectional view showing the configuration of the ultrasonic treatment tool of FIG. 6. As shown in FIG. 7, an air/water feed conduit 65 is provided to the tip side of the handpiece in the ultrasonic treatment tool 3B. The air/water feed conduit 65 extends to the vicinity of the jaw 34. Thereby, the treatment unit 12 is cooled by a fluid such as water, a gas, or the like running through the air/water feed conduit 65.

The air/water feed conduit 65 is connected to an air/water feed base 66. This air/water feed base 66 is detachably connected to the air/water feed cable 63.

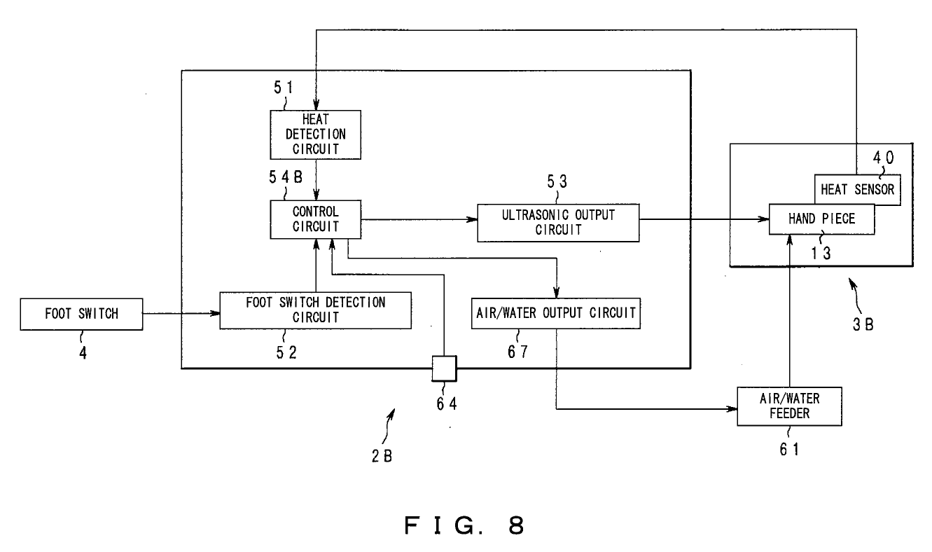

FIG. 8 is a block diagram showing a circuit of the ultrasonic surgical operation instrument of FIG. 6. As shown in FIG. 8, the instrument body 2B comprises an air/water output circuit 67 for controlling the drive of the air/water feeder 61.

The air/water output circuit 67 is controlled by a control circuit 54B. In other words, the air/water output circuit 67 receives the on/off signal and an air/water feed volume signal from the control circuit 54B and outputs a control signal for controlling the drive of the air/water feeder 61.

The control circuit 54B controls the air/water output circuit 67 in addition to the ultrasonic output circuit 53 on the basis of the on/off signal output from the foot switch detection circuit 52 and on the basis of the temperature information of the jaw 34 output from the heat detection circuit 51.

In other words, the control circuit 54B receives the input of the on/off signal from the foot switch 4 and receives the temperature information, outputs the on/off signal and the ultrasonic amplitude value signal to the ultrasonic output circuit 53, and also outputs the on/off signal and the air/water feed volume signal to the air/water output circuit 67.

In the second embodiment, a configuration is employed in which the air/water feeder 61 feeds air or water during a prescribed time period, interfacing with the off signal from the foot switch 4 when the air/water interface switch 64 is in the on state.

In the second embodiment, similarly to the first embodiment, a configuration is employed in which, during the prescribed time period, a temperature at which the treatment unit 12 does not incise the living tissue but coagulates the living tissue in the coagulation mode is maintained. The operation of the control circuit 54B will be explained by referring to the flowchart, which will be explained later.

The configurations other than this one are generally the same as the above first embodiment; accordingly, the explanation thereof is omitted.

The ultrasonic surgical operation instrument 1B configured as above can efficiently perform ultrasonic treatments such as incisions, coagulations and the like on living tissues.

The operator turns on the power switch 22 of the instrument body 2B. Then, the heat sensor 40 of the ultrasonic treatment tool 3B detects the temperature of the jaw 34 and starts transmitting the detected temperature information to the instrument body 2B. Also, the operator turns on the air/water interface switch 64. Thereby, the instrument body 2B can control the drive of the air/water feeder 61 when it is interfacing with the off signal of the foot switch 4.

Similarly to the case of the first embodiment, the operator places his/her fingers other than his/her thumb on the fixed handle 14a, places his/her thumb on the movable handle 14b, and grips the operation handle 14 of the ultrasonic treatment tool 3B while resisting the biasing force of the coil spring 38. Thereby living tissue is grasped between the jaw 34 and the tip treatment unit 32 of the ultrasonic probe 15.

In this state, the operator depresses the foot switch 4 and performs ultrasonic treatment on the living tissue grasped between the jaw 34 and the tip treatment unit 32 of the ultrasonic probe 15. The grasped living tissue receives an ultrasonic operation such as an incision, a coagulation, or the like via the friction heat between the living tissue itself and the tip treatment unit 32 that vibrates at high speed.

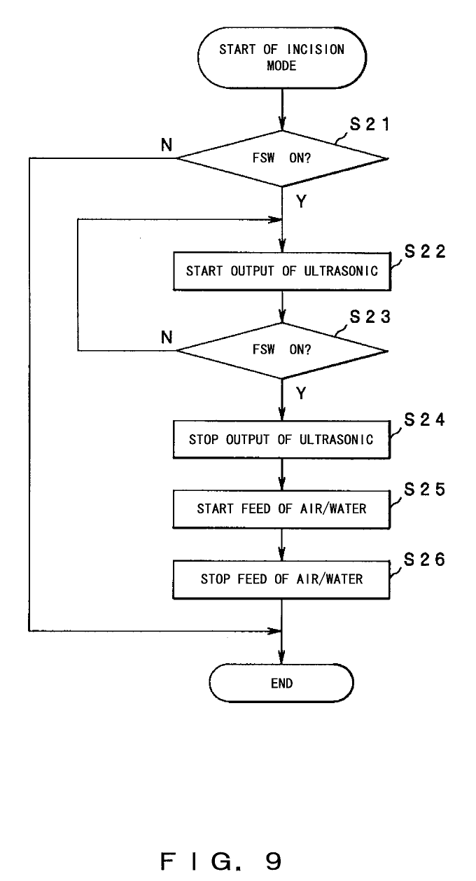

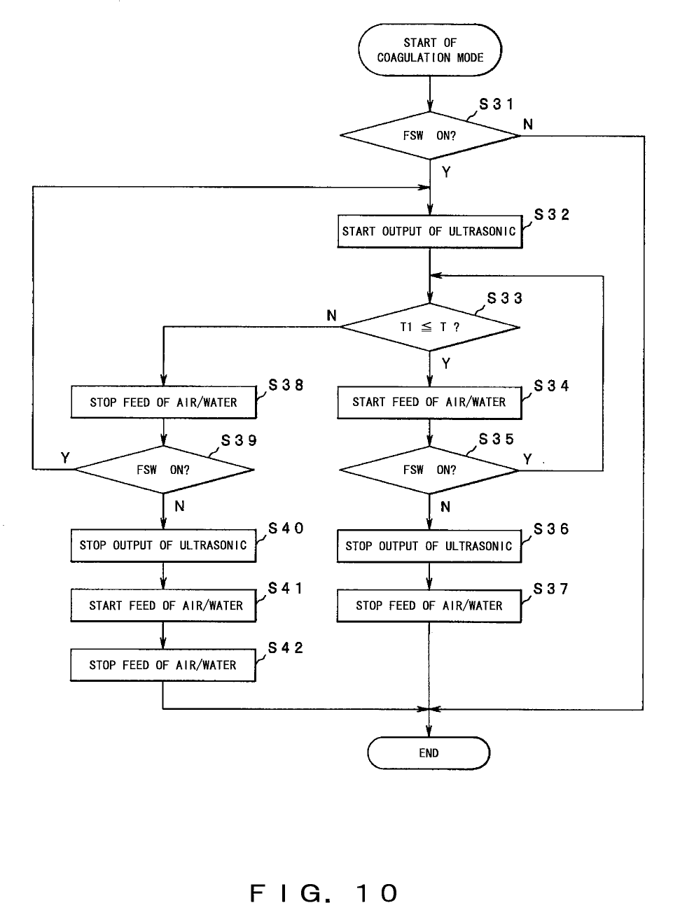

Then, the ultrasonic surgical operation instrument 1B operates according to the flowchart in FIG. 9 or the flowchart in FIG. 10. First, the incision mode is explained.

FIG. 9 is a flowchart showing the operation of the incision mode according to the second embodiment. When the operator depresses the incision switch 4a of the foot switch 4, the foot switch 4 outputs an incision-on signal via the foot switch cable 18.

In the ultrasonic surgical operation instrument 1B, the control circuit 54B starts control as shown in FIG. 9.

The control circuit 54B determines whether or not the incision switch 4a of the foot switch 4 is pressed (step S21). The control circuit 54B terminates the process shown in this flowchart when the incision-on signal is not received from the foot switch 4.

When the incision-on signal is received from the foot switch 4 via the foot switch detection circuit 52, the control circuit 54B outputs an on signal to the ultrasonic output circuit 53 and begins outputting the ultrasonic (step S22).

Then, the control circuit 54B receives the temperature information of the jaw 34 via the heat detection circuit 51. The control circuit 54B outputs the ultrasonic amplitude value signal on the basis of the temperature information received from the jaw 34. The ultrasonic output circuit 53 outputs the drive signal on the basis of the ultrasonic amplitude value signal from the control circuit 54B.

The drive signal from the instrument body 2B is transmitted to the ultrasonic vibrator 31 of the ultrasonic treatment tool 3B via the ultrasonic cable 16 and drives the ultrasonic vibrator 31.

Receiving the drive signal, the ultrasonic vibrator 31 generates ultrasonic vibrations. These ultrasonic vibrations are transmitted to the tip treatment unit 32 of the ultrasonic probe 15. The tip treatment unit 32 vibrates at high speed. The grasped living tissue is incised due to the friction heat between the living tissue itself and the tip treatment unit 32 vibrating at high speed.

During this incision, the operator keeps his/her foot on the incision switch 4a of the foot switch 4. When the operator lifts his/her foot away from the incision switch 4a of the foot switch 4, the foot switch 4 outputs an incision-off signal via the foot switch cable 18.

The control circuit 54B determines whether or not the incision switch 4a of the foot switch 4 is turned off (step S23). When the incision-off signal is not received from the foot switch 4, the control circuit 54B returns to step S22 and continues the above output of ultrasonic. When the incision-off signal from the foot switch 4 is received via the foot switch detection circuit 52, the off signal is output to the ultrasonic output circuit 53.

The ultrasonic output circuit 53 stops the output of the drive signal to the ultrasonic vibrator 31 when it receives the off signal from the control circuit 54B (step S24). The ultrasonic vibrator 31 stops the ultrasonic vibration.

Next, the control circuit 54B controls the air/water output circuit 67 and starts the air/water feed (step S25).

Then, the control circuit 54B generates the air/water feed volume signal and, on the basis of the temperature information received from the jaw 34, outputs the on signal and the air/water feed volume signal to the air/water output circuit 67 such that the air or water is fed for a prescribed time period.

On the basis of the on signal and the air/water feed volume signal from the control circuit 54B, the air/water output circuit 67 outputs the control signal such that the air or water is fed during the prescribed time period.

The control signal from the instrument body 2 is transmitted to the air/water feeder 61 via the air/water feed control cable 62 and drives the air/water feeder 61. The air/water feeder 61, receiving the control signal, feeds the air or water during the prescribed time period (for around three seconds, for example). The air/water feeder 61 feeds a fluid (such as a gas, water, or the like) to the ultrasonic treatment tool 3B.

In the ultrasonic treatment tool 3B, the fluid (such as a gas, water, or the like) mentioned above that is fed by the air/water feeder 61 via the air/water feed cable 63 is supplied to the vicinity of the jaw 34. The treatment unit 12 is cooled by the fluid (such as a gas, water, or the like) mentioned above that is supplied by the air/water feeder 61.

The control circuit 54B outputs the off signal to the air/water output circuit 67 after the prescribed time period.

The air/water output circuit 67 outputs the off signal to the air/water feeder 61 based on the off signal from the control circuit 54B. The air/water feeder 61 stops the feed of the air or water (step S26) and the ultrasonic treatment terminates.

Thereby, the ultrasonic surgical operation instrument 1B can decrease the temperature of the treatment unit 12 immediately after the incision of the living tissue and can prevent the heat denaturation of the surrounding living tissue that may result from being touched by the treatment unit 12 in the incision mode.

Next, the coagulation mode is explained.

FIG. 10 is a flowchart showing an operation of the coagulation mode according to the second embodiment. When the operator depresses the coagulation switch 4b of the foot switch 4 in order to turn it on, the foot switch 4 outputs a coagulation-on signal via the foot switch cable 18.

In the ultrasonic surgical operation instrument 1B, the control circuit 54B starts control as shown in FIG. 10. The control circuit 54B determines whether or not the coagulation switch 4b of the foot switch 4 is being pressed (step S31). The control circuit 54B terminates the process of this flowchart when the coagulation-on signal is not received from the foot switch 4 via the foot switch detection circuit 52.

When the coagulation-on signal is received from the foot switch 4 via the foot switch detection circuit 52, the control circuit 54B outputs an on signal to the ultrasonic output circuit 53 and starts outputting the ultrasonic (step S32).

Then, the control circuit 54B generates the ultrasonic amplitude value signal and outputs the signal to the ultrasonic output circuit 53. The ultrasonic output circuit 53 outputs the drive signal on the basis of the ultrasonic amplitude value signal from the control circuit 54B.

The drive signal from the instrument body 2B is transmitted to the ultrasonic vibrator 31 of the ultrasonic treatment tool 3B via the ultrasonic cable 16 and drives the ultrasonic vibrator 31.

Receiving the drive signal, the ultrasonic vibrator 31 generates ultrasonic vibrations. These ultrasonic vibrations are transmitted to the tip treatment unit 32 of the ultrasonic probe 15. The tip treatment unit 32 vibrates at high speed. The grasped living tissue starts to coagulate due to the friction heat between the living tissue itself and the tip treatment unit 32 vibrating at high speed.

Then, the control circuit 54B monitors the temperature T of the treatment unit 12 during the coagulation and controls cooling so that the temperature T of the treatment unit 12 is around a prescribed temperature T1 (around 120° C.).

In other words, the control circuit 54B determines whether or not the temperature T of the treatment unit 12 becomes equal to or higher than the prescribed temperature T1 (around 120° C.) during the coagulation (step S33). When the temperature T of the treatment unit 12 is equal to or higher than the temperature T1, the control circuit 54B controls the air/water output circuit 67 and starts the feeding of the air or water (step S34).

The control circuit 54B generates the air/water feed volume signal such that the volume of the fed air or water is the volume by which an increase in the temperature of the treatment unit 12 due to friction heat is prevented and outputs the on signal and the air/water feed volume signal to the air/water output circuit 67.

The air/water output circuit 67 outputs a control signal such that the volume of the fed air or water is the volume by which an increase in the temperature of the treatment unit 12 due to the friction heat is prevented on the basis of the on signal and the air/water feed volume signal from the control circuit 54B.

The control signal from the instrument body 2 is transmitted to the air/water feeder 61 via the air/water feed control cable 62 and drives the air/water feeder 61. The air/water feeder 61 feeds air or water after receiving the control signal. The air/water feeder 61 supplies a fluid (such as a gas, water or the like) to the ultrasonic treatment tool 3B.

In the ultrasonic treatment tool 3B, the fluid mentioned above that is fed by the air/water feeder 61 via the air/water feed cable 63 is supplied to the vicinity of the jaw 34.

Accordingly, in the ultrasonic treatment tool 3B, the treatment unit 12 is cooled by the fluid mentioned above that is supplied by the air/water feeder 61 and the temperature T of the treatment unit 12 becomes the temperature T1 (around 120° C.).

During this coagulation, the operator keeps his/her foot on the coagulation switch 4b of the foot switch 4. When the operator lifts his/her foot away from the coagulation switch 4b of the foot switch 4, the foot switch 4 outputs a coagulation-off signal via the foot switch cable 18.

The control circuit 54B determines whether or not the coagulation switch 4b of the foot switch 4 is in the on state (step S35). When the coagulation switch 4b of the foot switch 4 is in the on state, the control circuit 54B returns to step S33 and continues the above steps from step S33 to step S35 until the coagulation-off signal from the foot switch 4 is received via the foot switch detection circuit 52. When the coagulation-off signal from the foot switch 4 is received via the foot switch detection circuit 52, the control circuit 54B outputs the off signal to the ultrasonic output circuit 53.

The ultrasonic output circuit 53 stops the output of the drive signal to the ultrasonic vibrator 31 on the basis of the off signal from the control circuit 54 (step S36). The ultrasonic vibrator 31 stops the ultrasonic vibration.

The control circuit 54B outputs the off signal to the air/water output circuit 67 and the feeding of the air or water by the air/water feeder 61 is stopped (step S37). Then, the ultrasonic treatment terminates.

When the temperature T of the treatment unit 12 is lower than the temperature T1, the control circuit 54B outputs the off signal to the air/water output circuit 67 and stops the feeding of the air or water by the air/water feeder 61 (step S38).

The control circuit 54B determines whether or not the coagulation switch 4b of the foot switch 4 is in the on state (step S39). When the coagulation switch 4b of the foot switch 4 is in the on state, the control circuit 54B returns to step S32 and continues the above steps from step S32 to step S39 until the coagulation-off signal from the foot switch 4 is received via the foot switch detection circuit 52.

When the coagulation-off signal from the foot switch 4 is received via the foot switch detection circuit 52, the control circuit 54B outputs the off signal to the ultrasonic output circuit 53. The ultrasonic output circuit 53 stops the output of the drive signal to the ultrasonic vibrator 31 on the basis of the off signal from the control circuit 54 (step S40). The ultrasonic vibrator 31 stops the ultrasonic vibration.

Next, the control circuit 54B controls the air/water output circuit 67 in a manner similar to the explanation of the above incision mode and starts the feeding of the air or water (step S41).

Then, the control circuit 54B generates the air/water feed volume signal such that the air or water is fed during the prescribed time period in a manner similar to the above incision mode and outputs the on signal and the air/water feed volume signal to the air/water output circuit 67 on the basis of the temperature information received from the jaw 34.

The air/water output circuit 67 outputs the control signal via the air/water feed control cable 62 such that the air or water is fed during the prescribed time period, and drives the air/water feeder 61 on the basis of the on signal and the air/water feed volume signal from the control circuit 54B. The air/water feeder 61, receiving the control signal, feeds the air or water during the prescribed time period, and the treatment unit 12 is cooled.

The control circuit 54B outputs the off signal to the air/water output circuit 67 after the prescribed time period.

The air/water output circuit 67 outputs the off signal to the air/water feeder 61 on the basis of the off signal from the control circuit 54B. The air/water feeder 61 stops the feed of the air or water (step S42) and the ultrasonic treatment tool 3B terminates the ultrasonic treatment.

Thereby, the ultrasonic surgical operation instrument 1B can reliably perform coagulation on the living tissue without the operator controlling the energy applied to the living tissue in the coagulation mode by keeping the temperature of the treatment unit 12 at the prescribed temperature at which the living tissue will not be incised.

Accordingly, the ultrasonic surgical operation instrument 1B cools the treatment unit 12 and the living tissue via the air/water feed interfacing with the ultrasonic or via the air/water feed after the output of the ultrasound on the basis of the temperature information feedback, and can therefore control the increase in the temperature of the living tissue.

As a result of this, the ultrasonic surgical operation instrument 1B can attain the same effect as that attained in the first embodiment.

### Third Embodiment

In the above first embodiment, the temperature of the treatment unit 12 is kept at the prescribed temperature by controlling the ultrasonic amplitude value. In contrast, in a third embodiment, a configuration is employed in which the temperature of the treatment unit 12 is kept at the prescribed temperature by controlling the grasping force with which the living tissue is grasped. The other aspects of the configuration aside from this are generally the same as those of the first embodiment; accordingly, the explanations thereof are omitted and like members are denoted by like symbols.

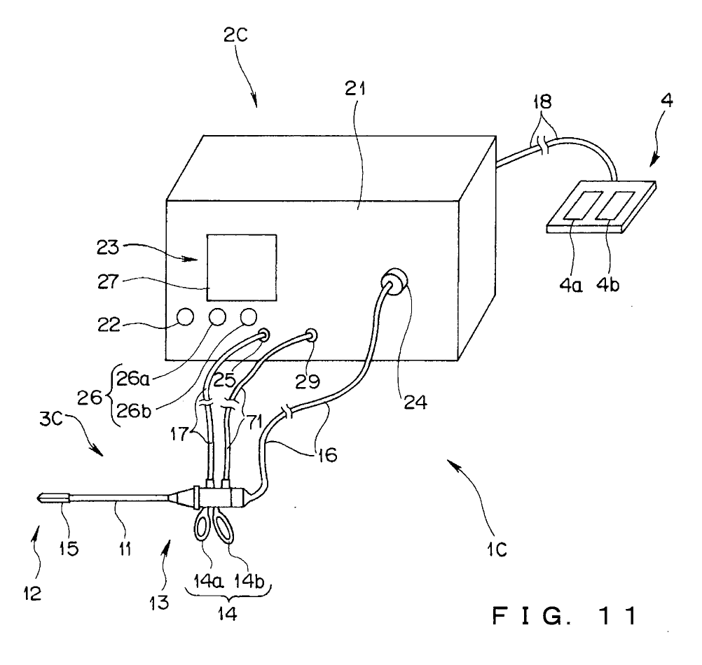

FIG. 11 is an overall view showing an ultrasonic surgical operation instrument according to the third embodiment. As shown in FIG. 11, the ultrasonic surgical operation instrument 1C according to the third embodiment has a configuration in which the ultrasonic surgical operation instrument 1C comprises an ultrasonic treatment tool 3C including electromagnets 72 (which will be described later) for adjusting the grasping force with respect to the grasped living tissue.

In the ultrasonic treatment tool 3C, an electromagnet output cable 71 extending from the tip side of the handpiece is connected to an electromagnet output cable connection unit 29 of the instrument body 2C.

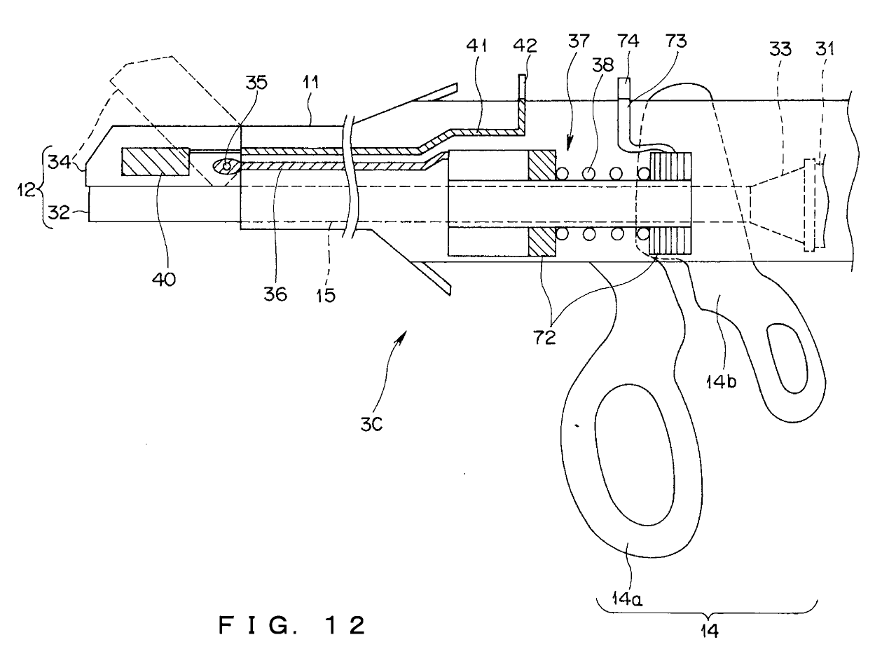

FIG. 12 is a schematic cross sectional view showing the configuration of the ultrasonic treatment tool shown in FIG. 11. As shown in FIG. 12, in the ultrasonic treatment tool 3C, a pair of electromagnets 72 are provided in the operation force adjustment unit 37 in a configuration in which the electromagnets 72 repel each other. A signal line 73 extending from the electromagnets 72 is connected to an electromagnet terminal 74. The electromagnet output cable 71 is detachably connected to this electromagnet terminal 74. The electromagnets 72 generate magnetic forces with current supplied from the instrument body 2C and generate repulsion by which the two electromagnets repel each other.

Thereby, in the ultrasonic treatment tool 3C, the operation rod 36 is moved forward via the repulsion of the electromagnets 72 in addition to the biasing force of the coil spring 38 and the jaw 34 is caused to close with respect to the tip treatment unit 32 of the ultrasonic probe 15.

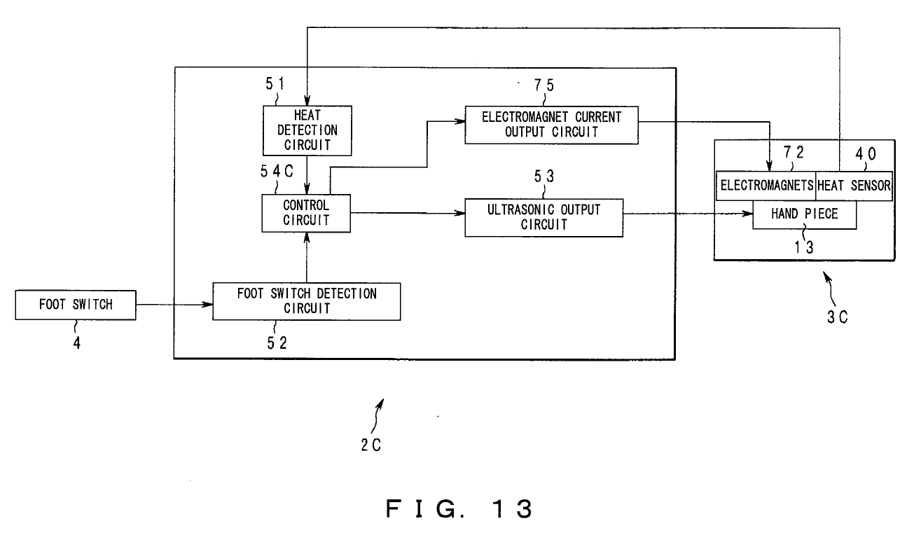

FIG. 13 is a block diagram for a circuit of the ultrasonic surgical operation instrument of FIG. 11. As shown in FIG. 13, the instrument body 2C comprises an electromagnet current output circuit 75 for controlling the drive of the electromagnets 72.

The electromagnet current output circuit 75 is controlled by a control circuit 54C. In other words, the electromagnet current output circuit 75 receives the on/off signal and current value signal from the control circuit 54C and outputs the current for controlling the drive of the electromagnets 72.

The control circuit 54C controls the electromagnets 72 in addition to the ultrasonic output circuit 53 on the basis of the on/off signal output from the foot switch detection circuit 52 and on the basis of the temperature information of the jaw 34 output from the heat detection circuit 51.

In other words, the control circuit 54C receives the temperature information and the input of the on/off signal from the foot switch 4 then outputs the on/off signal and the ultrasonic amplitude value signal to the ultrasonic output circuit 53 and also outputs the on/off signal and the current value signal to the electromagnet current output circuit 75.

In the third embodiment, similarly to the first embodiment, a configuration is employed in which the prescribed temperature is kept at a temperature at which the treatment unit 12 does not incise living tissue but coagulates the living tissue in the coagulation mode. The operation of the control circuit 54C will be explained later by referring to the flowchart.

The aspects of the configuration other than this aspect are generally the same as those in the above first embodiment; accordingly, the explanations thereof are omitted.

The ultrasonic surgical operation instrument 1C configured as above can efficiently perform ultrasonic treatments such incisions, coagulations and the like to living tissues.

The operator turns on the power switch 22 of the instrument body 2C. Then, the heat sensor 40 of the ultrasonic treatment tool 3C detects the temperature of the jaw 34 and starts transmitting the detected temperature information to the instrument body 2C via the heat sensor cable 17.

Similarly to the case of the first embodiment, the operator places his/her fingers other than his/her thumb on the fixed handle 14a, his/her thumb on the movable handle 14b, and grips the operation handle 14 of the ultrasonic treatment tool 3C while resisting the biasing force of a coil spring 38. Thereby, the operation force adjustment unit 37 pushes the operation rod 36 toward the tip portion while adjusting the operation force from the movable handle 14b in the ultrasonic treatment tool 3C.

The force transmitted by the operation rod 36 moving forward operates on the jaw 34 to close the jaw 34 that is rotating about the support shaft 35. Then, the jaw 34 closes with respect to the tip treatment unit 32 of the ultrasonic probe 15 while pinching living tissue between the jaw 34 itself and the tip treatment unit 32 of the ultrasonic probe 15. Thereby, the jaw 34 grasps the living tissue between the jaw 34 itself and the tip treatment unit 32.

In this state, the operator depresses the foot switch 4 and performs the ultrasonic treatment on the living tissue grasped between the jaw 34 and the tip treatment unit 32 of the ultrasonic probe 15. The grasped living tissue receives ultrasonic operations such as incisions, coagulations, or the like via the friction heat between the living tissue itself and the tip treatment unit 32 that vibrates at high speed.

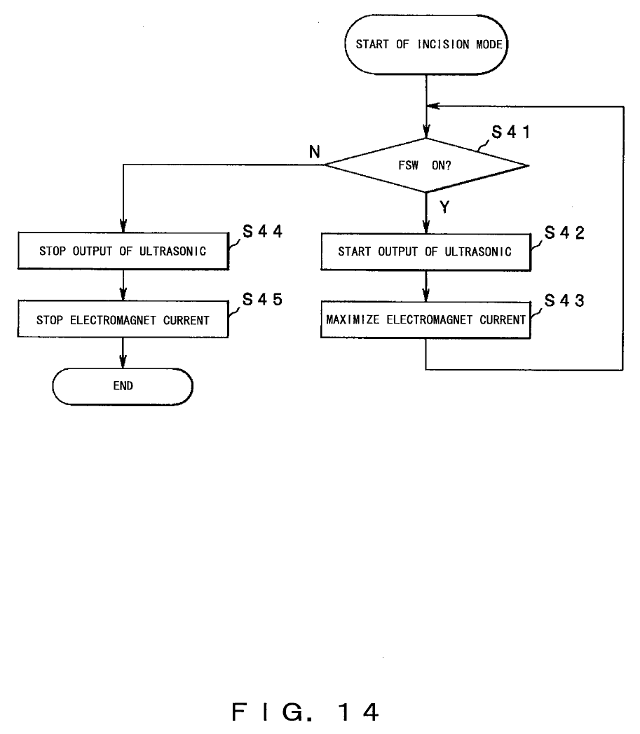

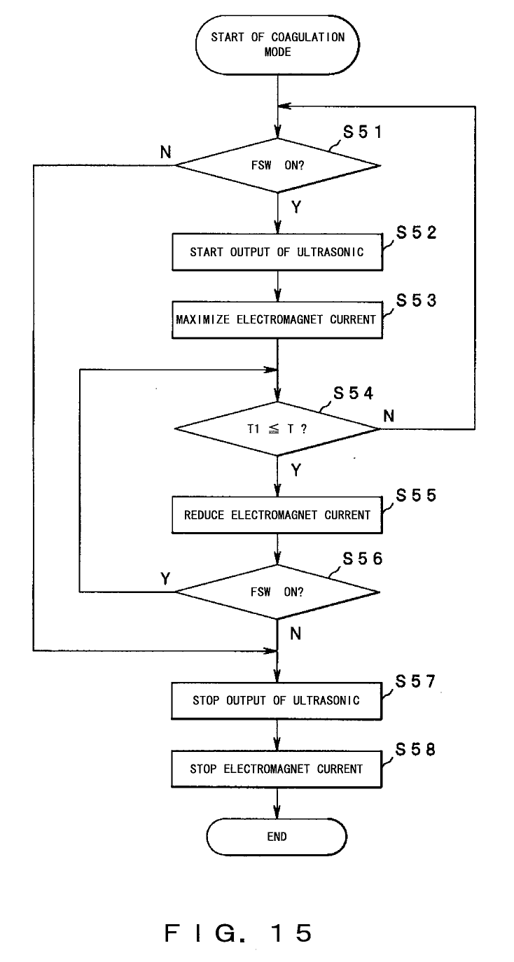

Then, the ultrasonic surgical operation instrument 1C operates according to the flowchart in either FIG. 14 or FIG. 15. First, the incision mode is explained.

FIG. 14 is a flowchart showing the operation of the incision mode according to the third embodiment. When the operator depresses the incision switch 4a of the foot switch 4, the foot switch 4 outputs an incision-on signal via the foot switch cable 18.

In the ultrasonic surgical operation instrument 1C, the control circuit 54C starts control as shown in FIG. 14.

The control circuit 54C determines whether or not the incision switch 4a of the foot switch 4 is being pressed (step S41). The control circuit 54C terminates the process of this flowchart when the incision-on signal is not received from the foot switch 4.

When the incision-on signal is received from the foot switch 4 via the foot switch detection circuit 52, the control circuit 54C outputs an on signal to the ultrasonic output circuit 53 and starts outputting the ultrasonic (step S42).

Then, the control circuit 54C receives the temperature information of the jaw 34 via the control circuit 54. The control circuit 54C outputs the ultrasonic amplitude value signal on the basis of the temperature information received from the jaw 34.

The ultrasonic output circuit 53 outputs the drive signal on the basis of the ultrasonic amplitude value signal from the control circuit 54C. The drive signal from the instrument body 2C is transmitted to the ultrasonic vibrator 31 of the ultrasonic treatment tool 3C via the ultrasonic cable 16 and drives the ultrasonic vibrator 31.

Receiving the drive signal, the ultrasonic vibrator 31 generates ultrasonic vibrations. These ultrasonic vibrations are transmitted to the tip treatment unit 32 of the ultrasonic probe 15. The tip treatment unit 32 vibrates at high speed. The grasped living tissue is incised due to the friction heat between the living tissue itself and the tip treatment unit 32 vibrating at high speed.

Then, the control circuit 54C controls the grasping force by controlling the electromagnet current output circuit 75. The control circuit 54C generates a current value signal such that the grasping force reaches its maximum possible force and outputs the on signal and the current value signal to the electromagnet current output circuit 75. The electromagnet current output circuit 75 outputs the maximum possible current on the basis of the on signal and the current value signal from the control circuit 54C (step S43).

The maximum possible current from the instrument body 2C is transmitted to the electromagnets 72 of the ultrasonic treatment tool 3C via the electromagnet output cable 71 and causes the electromagnets 72 to generate the maximum possible magnetic force. Then, the maximum possible repulsion force is generated. This repulsion force along with the biasing force of the coil spring 38 moves the operation rod 36 forward, and the jaw 34 is caused to close completely with respect to the tip treatment unit 32 of the ultrasonic probe 15.

Thereby, in the ultrasonic treatment tool 3C, the grasping force of the treatment unit 12 reaches its maximum possible force because the maximum possible repulsion force of the electromagnets 72 is effected; accordingly, the tip treatment unit 32 of the ultrasonic probe 15 strongly abuts against the living tissue.

Accordingly, the grasped living tissue is rapidly incised, receiving the full extent of ultrasonic vibrations from the tip treatment unit 32 of the ultrasonic probe 15.

During this incision, the operator keeps his/her foot on the incision switch 4a of the foot switch 4. When the operator lifts his/her foot away from the incision switch 4a of the foot switch 4, the foot switch 4 outputs an incision-off signal via the foot switch cable 18.

The control circuit 54C returns to step S41 and determines whether or not the incision switch 4a of the foot switch 4 is turned off. When the incision-off signal is not received from the foot switch 4, the control circuit 54C returns to step 42 and continues the above output of the ultrasonic. When the incision-off signal from the foot switch 4 is received via the foot switch detection circuit 52, the off signal is output to the ultrasonic output circuit 53.

The ultrasonic output circuit 53 stops the output of the drive signal to the ultrasonic vibrator 31 on the basis of the off signal from the control circuit 54C (step S44). The ultrasonic vibrator 31 stops the ultrasonic vibrations. Next, the control circuit 54C outputs the off signal to the electromagnet current output circuit 75. The electromagnet current output circuit 75 stops the supply of current on the basis of the off signal from the control circuit 54C (step S45). As a result of this, only the biasing force of the coil spring 38 constitutes the grasping force on the living tissue in the ultrasonic treatment tool 3C after this process and the ultrasonic treatment terminates.

Thereby, the ultrasonic surgical operation instrument 1C can provide the full extent of ultrasonic vibrations from the tip treatment unit 32 of the ultrasonic probe 15 to the grasped living tissue and can incise the living tissue rapidly in the incision mode.

Next, the coagulation mode is explained.

FIG. 15 is a flowchart showing the operation of the coagulation mode according to the third embodiment. When the operator depresses the coagulation switch 4b of the foot switch 4, the foot switch 4 outputs a coagulation-on signal via the foot switch cable 18.

In the ultrasonic surgical operation instrument 1C, the control circuit 54C starts control as shown in FIG. 15.

The control circuit 54C determines whether or not the coagulation switch 4b of the foot switch 4 is being pressed (step S51). The control circuit 54C terminates the process of this flowchart when the coagulation-on signal is not received from the foot switch 4 via the foot switch detection circuit 52.

When the coagulation-on signal is received from the foot switch 4 via the foot switch detection circuit 52, the control circuit 54C outputs an on signal to the ultrasonic output circuit 53 and starts outputting the ultrasonic (step S52).

Then, the control circuit 54C receives the temperature information of the jaw 34 via the heat detection circuit 51. The control circuit 54C outputs the ultrasonic amplitude value signal on the basis of the temperature information received from the jaw 34.

The ultrasonic output circuit 53 outputs the drive signal on the basis of the ultrasonic amplitude value signal from the control circuit 54C. The drive signal from the instrument body 2C is transmitted to the ultrasonic vibrator 31 of the ultrasonic treatment tool 3C via the ultrasonic cable 16 and drives the ultrasonic vibrator 31.

Receiving the drive signal, the ultrasonic vibrator 31 generates ultrasonic vibrations. These ultrasonic vibrations are transmitted to the tip treatment unit 32 of the ultrasonic probe 15. The tip treatment unit 32 vibrates at high speed. The grasped living tissue starts to coagulate due to the friction heat between the living tissue itself and the tip treatment unit 32 vibrating at high speed.

Then, the control circuit 54C controls the grasping force by controlling the electromagnet current output circuit 75. The control circuit 54C generates the current value signal such that the grasping force reaches its maximum possible force and outputs the on signal and the current value signal to the electromagnet current output circuit 75. The electromagnet current output circuit 75 outputs the maximum possible current on the basis of the on signal and the current value signal from the control circuit 54C (step S53).

The maximum possible current from the instrument body 2C is transmitted to the electromagnets 72 of the ultrasonic treatment tool 3C via the electromagnet output cable 71 and causes the electromagnets 72 to generate the maximum possible magnetic force. Then, the maximum possible repulsion force is generated. This repulsion force and the biasing force of the coil spring 38 move the operation rod 36 forward, and the jaw 34 is caused to close completely with respect to the tip treatment unit 32 of the ultrasonic probe 15.

Thereby, in the ultrasonic treatment tool 3C, the grasping force of the treatment unit 12 attains its maximum possible force because the maximum possible repulsion force of the electromagnets 72 is effected; accordingly, the tip treatment unit 32 of the ultrasonic probe 15 strongly abuts against the living tissue.

Accordingly, the grasped living tissue is rapidly incised, receiving the full extent of ultrasonic vibrations from the tip treatment unit 32 of the ultrasonic probe 15.

Then, the control circuit 54C monitors the temperature T of the treatment unit 12 during coagulation and controls the grasping force such that the temperature T of the treatment unit 12 is around a prescribed temperature T1 (around 120° C.).

In other words, the control circuit 54C determines whether or not the temperature T of the treatment unit 12 becomes equal to or higher than the prescribed temperature T1 (around 120° C.) during coagulation (step S54).

When the temperature T of the treatment unit 12 is lower than the temperature T1 (“N” in step S54), the control circuit 54 returns to step S51 and repeats the above steps from step S51 to step S54 until the temperature T of the treatment unit 12 becomes equal to or higher than the prescribed temperature T1 (around 120° C.).

When the temperature T of the treatment unit 12 is equal to or higher than the temperature T1 (“Y” at step S54), the control circuit 54 generates an ultrasonic amplitude value signal such that the grasping force is a force under which an increase in the temperature of the treatment unit 12 due to friction heat is prevented, and outputs the current value signal to the electromagnet current output circuit 75. The electromagnet current output circuit 75 outputs a reduced current on the basis of the current value signal from the control circuit 54C (step S55).

Thereby, in the ultrasonic treatment tool 3C, the grasping force is the force under which an increase in the temperature of the treatment unit 12 due to friction heat is prevented; accordingly, ultrasonic vibrations are provided with the tip treatment unit 32 of the ultrasonic probe 15 weakly abutting against the living tissue. The grasped living tissue coagulates due to heat denaturation caused by the friction heat between the living tissue itself and the tip treatment unit 32 vibrating at low speed without making an incision.

Accordingly, in the ultrasonic treatment tool 3C, the temperature T of the treatment unit 12 reaches the temperature T1 (around 120° C.). During this coagulation, the operator keeps his/her foot on the coagulation switch 4b of the foot switch 4. When the operator lifts his/her foot away from the coagulation switch 4b of the foot switch 4, the foot switch 4 outputs a coagulation-off signal via the foot switch cable 18.

The control circuit 54C determines whether or not the coagulation switch 4b of the foot switch 4 is in the on state (step S56). When the coagulation switch 4b of the foot switch 4 is in the on state, the control circuit 54C returns to step S53 and continues the above steps from step S53 to step S55 until the coagulation-off signal from the foot switch 4 is received via the foot switch detection circuit 52.

When the coagulation-off signal from the foot switch 4 is received via the foot switch detection circuit 52, the control circuit 54C outputs the off signal to the ultrasonic output circuit 53.

The ultrasonic output circuit 53 stops the output of the drive signal to the ultrasonic vibrator 31 on the basis of the off signal from the control circuit 54 (step S57). The ultrasonic vibrator 31 stops the ultrasonic vibrations.

Next, the control circuit 54C outputs the off signal to the electromagnet current output circuit 75. The electromagnet current output circuit 75 stops the supply of current due to the off signal from the control circuit 54C (step S58). As a result of this, only the biasing force of the coil spring 38 constitutes the grasping force on the living tissue in the ultrasonic treatment tool 3C after this process and the ultrasonic treatment terminates.

Thereby, the ultrasonic surgical operation instrument 1C can reliably perform coagulations on the living tissue without the operator controlling the energy applied to the living tissue in the coagulation mode by keeping the temperature of the treatment unit 12 at the prescribed temperature at which the living tissue is not coagulated.

Accordingly, the ultrasonic surgical operation instrument 1C can control the friction heat and can control the increase in the temperature of the treatment unit 12 and the living tissue by changing the grasping force during the output of the ultrasonic by using the temperature information as feedback.

As a result of this, the ultrasonic surgical operation instrument 1C can attain the same effect as that in the first embodiment.

In the third embodiment, the grasping force is controlled by using the repulsion of the electromagnets 72. However, the scope of the present invention is not limited to this; other configurations can be employed, including a configuration in which the grasping force is controlled by the attraction of the electromagnets 72 and a configuration in which the grasping force is controlled by the combination of the repulsion and the attraction.

The scope of the present invention is not limited to the above embodiments, and various alternations, modifications, and the like are allowed without changing the spirit of the present invention.

As above, the ultrasonic surgical operation instrument according to the present invention attains the effect that the energy applied to the living tissue can be controlled without the consciousness of the operator and the temperature with respect to the living tissue can be prevented from becoming too high.

What is claimed is:

1. An ultrasonic surgical operation instrument, comprising:
a treatment unit for performing treatments on a living tissue using ultrasonic vibrations;
a heat sensor that is disposed in the treatment unit and that detects a temperature of the treatment unit; and
a control unit for controlling the temperature of the treatment unit on the basis of the temperature information detected by the heat sensor.

2. The ultrasonic surgical operation instrument according to claim 1, wherein:
on the basis of the temperature information detected by the heat sensor, the control unit controls the temperature of the treatment unit such that the temperature is kept at the prescribed temperature at which living tissue coagulates.

3. The ultrasonic surgical operation instrument according to claim 2, wherein:
on the basis of the temperature information detected by the heat sensor, the control unit controls the temperature of the treatment unit by controlling an amplitude of the ultrasonic output provided to the treatment unit, by controlling cooling by feeding air or water to the treatment unit, or by controlling a grasping force of the treatment unit.

4. An ultrasonic surgical operation instrument, comprising:
a treatment unit that includes an ultrasonic probe for giving treatment to a living tissue using ultrasonic vibrations and a jaw for grasping the living tissue between the jaw and the ultrasonic probe;
a heat sensor that is disposed in the treatment unit and that detects a temperature of the treatment unit; and
a control unit for controlling the temperature of the treatment unit on the basis of the temperature information detected by the heat sensor.

5. The ultrasonic surgical operation instrument according to claim 4, wherein:
the control unit controls the temperature of the treatment unit such that the temperature is kept at the prescribed temperature at which a living tissue coagulates on the basis of the temperature information detected by the heat sensor.

6. The ultrasonic surgical operation instrument according to claim 5, wherein:
on the basis of the temperature information detected by the heat sensor, the control unit controls the temperature of the treatment unit by controlling an amplitude of the ultrasonic output for driving the ultrasonic probe, by controlling cooling by feeding air or water to the treatment unit, or by controlling the grasping force between the ultrasonic probe and the jaw for grasping living tissue.
